(19) United States
(12) Patent Application Publication
(10) Pub. No.: US 2023/0149144 A1
(43) Pub. Date: May 18, 2023

(54) ELECTRODE APPLICATORS FOR CONJUNCTIVE USE IN A DENTAL IMPLANT TREATMENT SYSTEM

(71) Applicant: Garwood Medical Devices, LLC, Buffalo, NY (US)

(72) Inventors: Timothy M. Kopera, Lancaster, NY (US); Jackson G. Hobble, Buffalo, NY (US); Wayne D. Bacon, Buffalo, NY (US); Brian R. Peterson, East Aurora, NY (US)

(21) Appl. No.: 17/909,628

(22) PCT Filed: Jul. 7, 2020
(86) PCT No.: PCT/US2020/041022

§ 371 Date: Sep. 6, 2022

Related U.S. Application Data
Application No. PCT/US2020/041022 is a continuation-in-part of application No. 16/884,664, filed on May 27, 2020.
(60) Provisional application No. 62/984,332, filed on Mar. 3, 2020.

(51) Int. Cl. A61C 19/06; A61N 1/05; A61N 1/20

(52) U.S. Cl. CPC A61C 19/06; A61N 1/205; A61N 1/0548

(57) ABSTRACT

An apparatus for use with a treatment system to disrupt bacteria from a metallic dental implant includes apparatus configured for mechanical and electrical connection to a device capable of producing an electrical stimulation voltage, and in which a connective body is further configured for attachment to the mouth of a patient and including at least one metal contact. The apparatus can include a mouth guard having at least one integrated or releasably attachable metal contact. Alternatively, at least one clip member can be attached, the clip member having a metal contact portion. The apparatus can be used in conjunction with any electrode of a treatment system in which the electrode is disposed within the mouth of the patient or external.

### CROSS REFERENCE TO PRIOR APPLICATIONS

This application is a national stage application under 35 U.S.C. §371 of International Application No. PCT/US2020/041022, filed Jul. 7, 2020, which claims priority under applicable portions of 35 U.S.C. §§119 and 120 to U.S. Pat. Application Serial No. 16/884,664, filed on May 27, 2020 and U.S. Pat. Application Serial No. 62/984,332, filed Mar. 3, 2020, the entire contents of each application being herein incorporated by reference.

### TECHNICAL FIELD

The application is directed generally to the field of treatment systems used to disrupt bacteria from surgically implanted devices, and more specifically to supporting and configuring electrodes used in systems for treating infected metal dental implants.

### BACKGROUND

Metal implants are used in patients with many different injuries or medical problems. For example, various orthopedic devices such as knee, hip or shoulder joint replacements can be surgically implanted. Similarly, metal implants may be used for any individual that needs to replace a tooth in a dental procedure. Dental implants are commonly used to completely replace a tooth. More specifically, dental implants are made up of three (3) components; namely, a metallic post that is osseointegrated to the jaw bone of the patient, an abutment extending from the metallic post, and a prosthetic tooth (a dental crown), in which the latter can be made from an electrically non-conductive material, which is disposed over the abutment.

One potential problem with metal implants in general is that they tend to allow for the growth of bacteria on the surface. This may increase the patient’s risk for an infection. This issue is especially prevalent in the mouth due to a large bacterial presence. As bacteria colonize upon foreign surfaces such as metal, biofilms are formed. Biofilms are protective extracellular matrix materials that encapsulate bacterial colonies onto a surface and protect them. Biofilms can be 500-5000 times more resistant to antibiotics than common planktonic bacteria because the antibiotics cannot penetrate the biofilm. Statistically, a significant percentage (greater than 14 percent) of dental implants acquire periimplantitis, or bacterial infection of the implant that can cause complications with implant loosening, gum and bone loss.

To decrease the risk of infection, electrodes can provide electrical stimulation to disrupt the growth of bacteria. It has been shown in scientific literature that the application of a suitable cathodic current to metal samples create chemical reactions at the surface of the implant that can disrupt and kill bacterial biofilms that exist on the metal.

For electrochemical processes to occur, there must be an anode and a cathode within an electrolyte solution. The anode is a metallic surface where oxidative reactions occur, and the cathode is another metallic surface where reduction reactions occur. A reduction reaction is essentially when the material of interest gains electrons and thereby decreases the oxidation state of the molecules. The electrolyte that the electrodes each reside in provides the electrical connection by facilitating the flow of electrons shuttled by ion carriers such as sodium or potassium ions. Electrons are driven from the anode to the cathode through the electrical path via a potentiostat. A potentiostat is an instrument used to drive current from a counter electrode to a working electrode in order to keep the voltage on the working electrode at a constant value compared to a stable reference electrode. A treatment technique based on cathodic voltage controlled electrical stimulation (CVCES) is described in U.S. Pat. No. 9,616,142, herein incorporated in its entirety by reference. In this treatment technique, the anode represents the counter electrode and the cathode represents the working electrode. Using a potentiostat, a user can dictate which electrochemical process is occurring on the working electrode and at what rate it occurs simply by adjusting the applied voltage parameters with respect to a separate reference electrode. The cathodic reactions occurring at the working electrode produce hydroxide ions, resulting in an alkaline pH at on the implant surface while also producing different reactive oxidative chemical species that are bactericidal for existing biofilms.

In a research setting, the above treatment technique has been shown as a way to fight bacterial biofilm infections on metallic implants in the most minimally invasive way possible. In this setting, the patient’s bodies can act as an electrochemical cell by using the metal implant as the cathode and the counter electrode as the anode. The treatment system uses the electrochemical properties of the two electrodes in a DC circuit to chemically kill the biofilm, which means the electrodes must be submersed or contacting an electrolyte that transports the electrical energy through chemical reactions to the other electrode. Human bone and soft tissue provide this electrolyte media for conduction, and thus the complete surface area of the dental implant embedded in the bone receives treatment. Full surface treatment optimizes effectiveness against biofilm infections.

For dental implants in particular, an electrical connection to the implant can be difficult due to the non-conductive crown or crown coating that sits on top of the metal post and abutment, and above the gum line. Various approaches to electrically connect to the metal post for treatment include removing the crown or using a needle to pierce through the gum. Each of these approaches are impractical and inconvenient, as well as uncomfortable for the patient. Moreover, the design of any dental apparatus or medical device must highly consider patient safety and comfort. The treatment device must be both efficient and non-toxic relative to the patient.

### BRIEF DESCRIPTION

As described above, it has been demonstrated that applying cathodic voltages to a metallic material kills any form of bacterial biofilm that exists on the metal. When applying this therapy to an infected dental implant, it is preferable to keep the prosthetic crown attached, as opposed to alternative attachment mechanisms that need to connect to the implant abutment directly with no crown. The disclosed apparatus provides means of contact to exposed portions of the abutment, or specialized electrical contact points provide on a specialized crown, such as that described in U.S. Pat. Application No. 16/884,664, herein incorporated by reference or other variants. The apparatus and related method of the present invention also involves novel features that optimize the cathodic voltage system for patient safety in the oral cavity.

The disclosed invention presents a novel apparatus to both make electrical contact with an exposed dental abutment or a specialized crown with exposed metal, as well as novel embodiments of the application of the counter electrode (anode) and the reference electrode within the mouth. Optimal application of the implant (working electrode) connector, the counter electrode, and the reference electrode allows for efficient and concise connection to an external voltage source. This system and related method allows the physician to treat the fully bone-embedded implant surface, while still maintaining patient safety parameters and its minimally invasive profile.

The present invention relates to the use of voltage controlled electrical treatment to metallic surfaces as a method to prevent and eradicate microbial colonization on the surface, such as in the case of periimplantitis, common to dental implants. This invention is implemented when a DC electrical current is applied to a metallic implant. The system requires at least two (2) electrodes, but can also utilize three (3) or more electrodes. Specifically and in the case of three (3) electrodes, a counter electrode, a working electrode, and a reference electrode are provided in which the counter electrode delivers the current to the working electrode in order to maintain a steady DC potential with respect to the stable reference electrode. In the case of a dental implant, the metallic surface of the implant post and abutment act as the working electrode.

Novel mechanisms are disclosed to reliably attach electrodes to the dental implant and the tissue within the mouth to enable the chemical reaction for biofilm treatment to proceed safely and effectively. The herein disclosed apparatus provides a novel way of incorporating all elements necessary to provide an effective cathodic voltage electrical stimulation to a dental implant while maintaining patient safety and optimizing the treatment of the biofilm infection. These elements include the various electrodes of the treatment system, as well as physical applicator apparatus as described herein.

Therefore and according to one aspect, there is provided an apparatus for use with a treatment system that disrupts bacteria from a metallic dental implant, the treatment system comprising a device capable of producing a stimulation voltage, a counter electrode, and a working electrode each coupled to the device capable of producing the stimulation voltage. The apparatus comprises a connective body configured for connection to the device capable of producing a stimulation voltage and having at least one feature configured for attachment to the mouth of a patient, the body including at least one metal contact configured for electrical contact with an exposed metal area of the metallic dental implant as the working electrode.

The connective body according to at least one embodiment can comprise a mouth guard, which is shaped and configured to fit over the teeth and gums of a patient. In at least one version, the at least one metal contact is integrated into a wall of the mouth guard. In another version, the at least one metal contact can be releasably attached to a wall of the mouth guard.

The mouth guard can include a grounding plate imbedded in the wall of the mouth guard, wherein the at least one metal contact is formed on a clip member that is releasably attachable to the mouth guard. According to at least one version, the apparatus can comprise two or more clip members that can be releasably disposed on the mouth guard.

In an embodiment, the at least one metal contact of the mouth guard is biased into contact with an exposed metal area of at least one dental implant when attached to the mouth of a patient. The biasing can occur in a number of ways. For example and according to one version, the at least one metal contact can comprise a section of a conductive sponge or steel wool. According to another version, the at least one metal contact comprises a spring section of steel or other conductive material.

According to at least one other version, the connective body comprises at least one clip member configured for direct attachment to at least one tooth of a patient. The at least one clip member can include a torsional spring configured to bias the at least metal contact into contact with an exposed metal area of at least one dental implant when attached. The at least one clip member can further comprise a soft pad opposite the at least one metal contact configured for contacting the tooth of a patient when attached.

According to another aspect of the invention, there is described an apparatus for use with a treatment system that disrupts bacteria from a metallic dental implant, the treatment system comprising a device capable of producing a stimulation voltage, a counter electrode, and a working electrode each coupled to the device capable of producing the stimulation voltage, the working electrode comprising the metallic dental implant, the apparatus comprising the counter electrode including a connective body adapted for attachment to the gum line of the patient.

According to at least one version, the connective body comprises a flexible member configured to wrap about the teeth and gums of at least a portion of the mouth of a patient, the flexible member comprising a conductive anodic layer.

According to another version, the apparatus comprises a conductive member disposed within a container external to the mouth of the patient, the container containing a conductive fluid that is fluidically connected to an applicator disposed within the mouth of the patient. The conductive fluid is preferably neutral to basic in pH, wherein the container is configured to deliver conductive fluid to the connective body. In at least one version, the connective body supports at least one cotton roll that is saturated by the conductive fluid wherein the conductive fluid is transferred using a hollow tube disposed between the container and at least one cotton roll.

According to yet another aspect, there is provided a treatment system to disrupt bacteria from a metallic dental implant comprising a device capable of producing a cathodic stimulation voltage, a working electrode that comprises the metallic dental implant and a counter electrode. Each of the counter electrode and working electrode are connected via a circuit to the device capable of producing a stimulation voltage. The counter electrode comprises a container retaining a conductive fluid and an electrically conductive member, the container being connected to the device capable of producing the stimulation voltage. The system further comprises a connective body fluidically coupled to the container and the gum interface of a patient in relation to the metallic dental implant, the connective body being configured to receive conductive fluid and current created by the device capable of producing the stimulation voltage.

According to another aspect, there is provided an apparatus for use with a treatment system that disrupts bacteria from a metallic dental implant, the treatment system comprising a device capable of producing a stimulation voltage, a counter electrode, and a working electrode each coupled to the device capable of producing the stimulation voltage, the apparatus comprising a mouth guard configured for attachment to the mouth of a patient and having at least one metal contact configured for electrical contact with an exposed metal area of the metallic dental implant as the working electrode.

According to yet another aspect, there is provided an apparatus for use with a treatment system that disrupts bacteria from a metallic dental implant, the treatment system comprising a device capable of producing a stimulation voltage, a counter electrode, and a working electrode each coupled to the device capable of producing the stimulation voltage, the apparatus comprising at least one clip member configured for attachment to at least one tooth of a patient.

According to yet another aspect, there is provided a treatment system for disrupting bacteria from a metallic dental implant, the system comprising a device capable of providing a cathodic stimulation voltage, a working electrode capable of making electrical contact with at least one metallic dental implant; and a counter electrode electrically coupled with the gum line of a patient in proximity to the at least one metallic dental implant, each of the working and counter electrode being coupled in a circuit.

In at least one embodiment, the treatment system further comprises a reference electrode coupled to the circuit, the reference electrode being configured for monitoring treatment of the at least metallic dental implant.

According to at least one embodiment, the working electrode further comprises a connective body configured for attachment to the mouth of a patient, the connective body including at least one metal contact for engaging an exposed metal area of the at least one metallic dental implant. In at least one version, the connective body comprises a mouth guard shaped and configured to fit over the teeth and gums of a patient in which the at least one metal contact is integrated into a wall of the mouth guard or alternatively the least one metal contact is releasably attachable to a wall of the mouth guard.

A grounding plate is imbedded in the wall of the mouth guard wherein the at least one metal contact can be formed on a clip member that is releasably attachable to the mouth guard. According to at least one version, two or more clip members are configured to be releasably disposed on the mouth guard.

In at least one embodiment, the connective body comprises at least one clip member configured for attachment to at least one tooth of a patient. Means are provided for biasing the at least one metal contact into contact with the exposed metal area of at least one dental implant when the connective body is attached to the mouth of a patient. In one version, the at least one metal contact can comprise a section of conductive sponge or steel wool. In another version, the at least one metal contact can comprise a spring section made from conductive material.

In at least one version, the clip member can comprise a torsional spring configured to bias the at least metal contact into contact with an exposed metal area of at least one dental implant when attached. The at least one clip member can further comprise a soft pad opposite the at least one metal contact configured for contacting the tooth of a patient when attached.

The metallic dental implant comprises a crown disposed over a post, wherein the crown includes a metallic core having an exposed end in electrical contact with a metallic post fused to the jaw of the patient.

According to another embodiment, the connective body can comprise a flexible member configured to wrap about the teeth and gums of at least a portion of the mouth of a patient, the flexible member comprising a conductive anodic layer. The flexible member can further comprise a conductive mesh layer disposed between the conductive anodic layer and an exterior adhesive layer. In addition, a hydrogel layer having a buffered agent is disposed between the conductive anodic layer and exterior adhesive layer.

According to another embodiment, the counter electrode comprises at least one conductive member disposed within a container external to the mouth of the patient, the container containing a conductive fluid that is fluidically connected to the connective body disposed within the mouth of the patient. In some versions, the conductive fluid is neutral to basic in pH.

The container is configured to deliver conductive fluid to the connective body in which conductive fluid can be delivered to the connective body by at least one hollow tube and wherein the connective body includes at least cotton roll configured to receive conductive fluid from the container.

An advantage is that the herein described apparatus provides alternative means for dentists to treat infections that statistically affect roughly 14% of all people who receive a dental implant in a manner that is very minimally invasive.

The novel embodiments of the herein described dental implant treatment system that include abutment and crown contact mechanisms, as well as novel counter electrode embodiments give the physician optimal ability to apply a cathodic voltage system that can effectively disrupt and eliminate biofilm from a dental implant without removing the crown. A distinct differentiator from alternative dental treatment techniques is that this system promotes conduction over the entire bone-embedded surface of the dental implant, not just within the abscess pocket. This is key, especially in regard to dental implant posts. The posts are manufactured to have a very rough, coarse microsurface to promote osseointegration. One issue this surface can create is that bacteria are able to “hide” within the crevices of the microstructure, even when bone matrix are apparently grown into the surface. The approach and design of this novel system allows for thorough treatment of all microstructures in the metal, even with bone present, to eliminate all bacteria from those location. It has been found in scientific literature that at optimizes treatment parameters, matrix embedded bone cells that are local to the reaction are not affected to a high degree.

These and other technical features and advantages will be readily apparent from the following Detailed Description, which should be read in conjunction with the accompanying drawings.

### BRIEF DESCRIPTION OF THE DRAWINGS

FIG. 1 (a) is a side elevational view of a dental implant that enables access by a treatment system without removal of the crown;

FIG. 1(b) is a sectioned view of the dental implant of FIG. 1(a) taken through Section A-A;

FIG. 1 (c) is a partially exploded view of the dental implant of FIGS. 1(a) and 1(b);

FIG. 2 (a) is schematic diagram of an exemplary CVCES treatment system;

FIG. 2(b) is a schematic diagram of another exemplary CVCES treatment system that includes working, counter and reference electrodes;

FIG. 2(c) is a schematic diagram of yet another CVCES treatment system incorporating a plurality of electrodes;

FIG. 3 (a) is a top perspective view of an apparatus for use in a CVCES treatment system, including but not limited to those shown in FIGS. 2(a) - 2(c), which is made in accordance with aspects of the invention;

FIG. 3(b) is a top perspective view of the apparatus of FIG. 3(a), including a contact portion made in accordance with aspects of the invention;

FIGS. 3(c) and 3(d) are top perspective views of the apparatus of FIG. 3(a), including contact portions made in accordance with alternative aspects of the invention;

FIG. 4(a) depicts a top perspective view of an apparatus in accordance with an exemplary embodiment, including one or more releasably attachable contacts, according to one configuration;

FIG. 4(b) depicts the top perspective view of the apparatus of FIG. 4(a), including the one or more releasable contacts as disposed in another configuration;

FIG. 4(c) is a partial sectioned view depicting the attachment of a releasable contact to the apparatus of FIGS. 4(a) and 4(b);

FIG. 4(d) depicts an enlarged view of the releasable contact attachment of FIG. 4(c);

FIG. 4(e) is a partial perspective view of the apparatus of FIGS. 4(a) - 4(d), depicting another alternative placement of the releasable contacts;

FIG. 4(f) is a perspective view of the apparatus of FIGS. 4(a) - 4(e), depicting the releasable attachability of the contacts;

FIG. 5(a) is a perspective view of an apparatus made in accordance with other aspects of the invention and as attached to a patient;

FIG. 5(b) is an elevational view of the apparatus of FIG. 5(a), showing a contact portion made in accordance with an exemplary embodiment;

FIG. 5(c) is a top perspective view of the apparatus of FIGS. 5(a) and 5(b);

FIGS. 5(d) and 5(e) are perspective view of the apparatus of FIGS. 5(a) - (c), showing alternative contact portions made in accordance with aspects of the invention;

FIG. 6(a) is a perspective view of an apparatus made in accordance with another exemplary embodiment;

FIG. 6(b) is an exploded view of an electrode made for use in the apparatus of FIG. 6(a);

FIGS. 7 and 8 are schematic views of an external electrolytic delivery system in accordance with aspects of the invention; and

FIGS. 9(a) - 9(c) are views of the placement of cotton or similar material into the oral cavity of a patient for use with the external electrolytic delivery system of FIGS. 7 and 8.

### DETAILED DESCRIPTION

The present disclosure provides several novel embodiments of apparatus used in conjunction with treatment systems in order to disrupt and remove biofilms from a metallic dental implant. The treatment systems discussed utilize electrochemical stimulation therapy through an established electrical connection to the metallic dental implant and application of a suitable cathodic voltage. Connection is described in the following embodiments to a specific form of dental implant that enables treatment without requiring removal of the crown portion. It will be readily apparent, however, that this implant is an example and the herein described apparatus can be adapted for use with other types of dental implants. The novel apparatus designs that are discussed improve the overall ease and efficiency of treating metallic dental implants with suitable cathodic stimulation voltages.

As a matter of background and when a patient has a tooth that needs to be removed, the standard procedure is to replace that tooth with a dental implant. The dental implant typically is made up of three (3) main components that include a metallic post that is osseointegated to the jaw bone of a patient and a prosthetic tooth (crown) that is placed over an abutment of the metallic post. The tooth and roots are extracted and the bone is reamed to properly fit the metal post. A healing abutment is placed until the site is sufficiently healed. The healing abutment is then removed, and another metal abutment is screwed onto the post. The prosthetic crown can then be adjoined to the abutment. The crown typically has a hollowed core that allows the abutment to be press fit inside. It is also common to have an abutment that screws into the crown itself. The screw hole is typically at the top of the tooth and then is filled once the complete implant is in place. In many cases of this implantation, especially if there is an infection present that causes recession of tissue, the metal abutment is visibly exposed at the base. This situation provides a means of directly contacting with the abutment via a working electrode connection in order to provide cathodic stimulation for treatment of the infection and to disrupt biofilm layers.

The herein described system and method relies upon the introduction of an electrical current to an electrochemical cell. As a matter of background and for electrochemical (redox) processes to occur, there must be an anode and a cathode within an electrolyte solution. The anode is a metallic surface where oxidative reactions occur, and the cathode is another metallic surface where reduction reactions occur. A reduction reaction occurs when the material of interest gains electrons and thereby decreases the oxidation state of the molecules. The electrolyte that the electrodes each reside in provides the electrical connection by facilitating the flow of electrons shuttled by ion carriers, such as electrolytic sodium or potassium ions. Electrons are driven from the anode to the cathode through the electrical path via a potentiostat or similar device. More specifically, a potentiostat is an instrument used to drive current from a counter electrode to a working electrode in order to keep the voltage on the working electrode at a constant value compared to a stable reference electrode. One such procedure used for the treatment of biofilms on a metallic implant is described in U.S. Pat. No. 9,616,142, the entire contents of which are herein incorporated by reference.

According to this treatment procedure, the anode represents the counter electrode and the cathode represents the working electrode. Using a potentiostat, a user can dictate which electrochemical process is occurring on the working electrode and at what rate the process occurs simply by adjusting the applied voltage parameters with respect to a separate reference electrode. The cathodic reactions occurring at the working electrode produce hydroxide ions, resulting in an alkaline pH at on the implant surface, while also producing different reactive oxidative chemical species that are bactericidal for existing biofilms.

In a research setting, the above-noted technique has been shown as a way to fight bacterial biofilm infections on metallic implants in the most minimally invasive way possible. In this setting, the patient’s body can act as an electrochemical cell by using the metal implant as the cathode and the counter electrode as the anode. It has been shown that the above techniques can be used for the treatment of various orthopedic implants, including metallic dental implants.

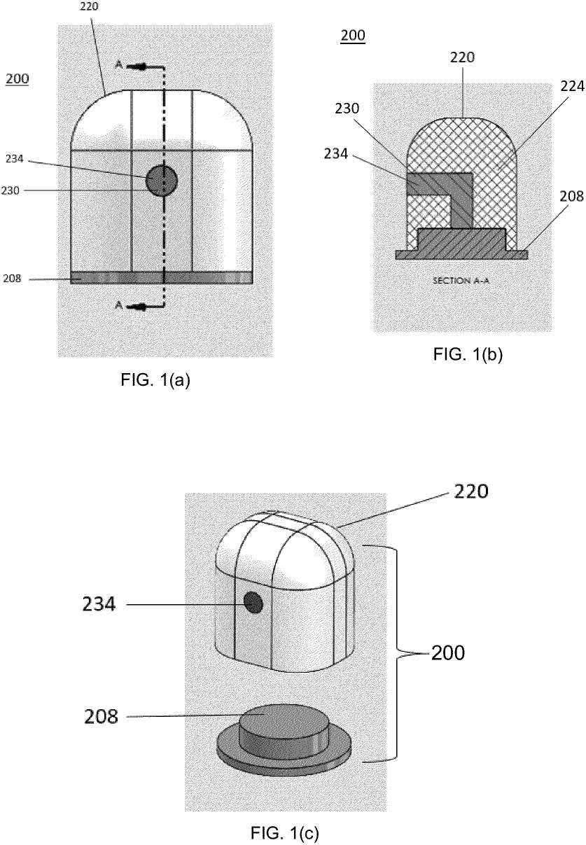

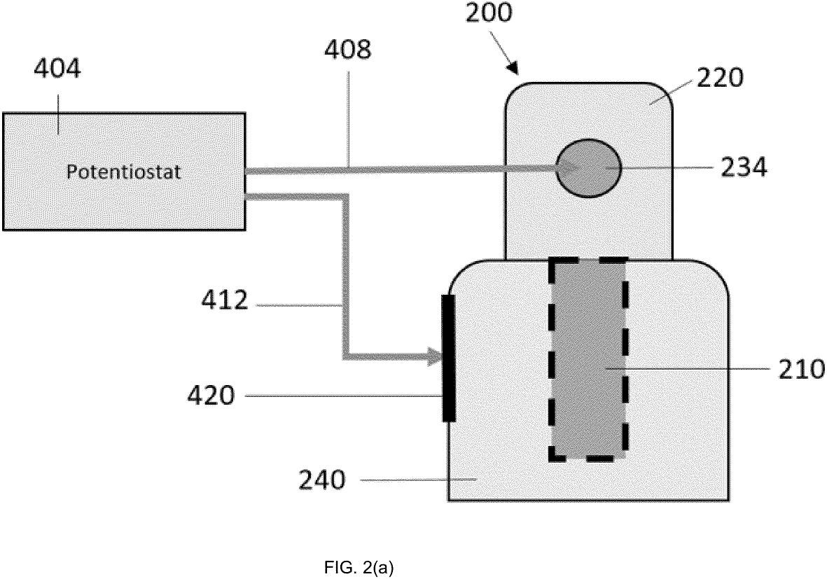

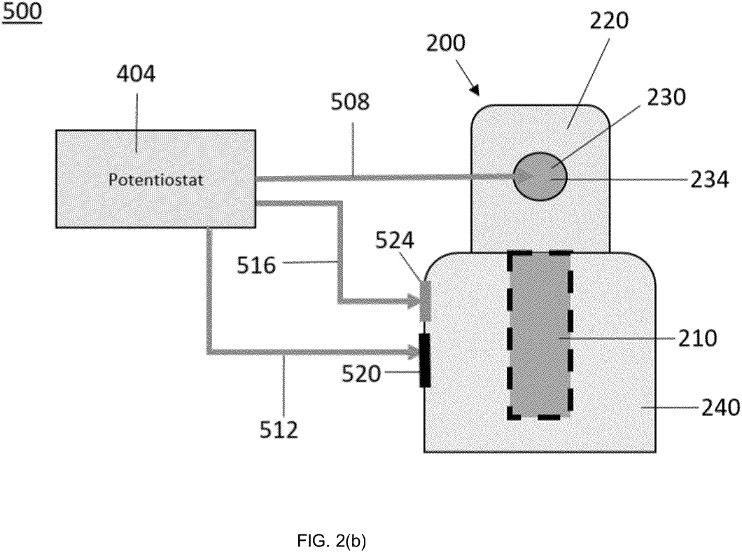

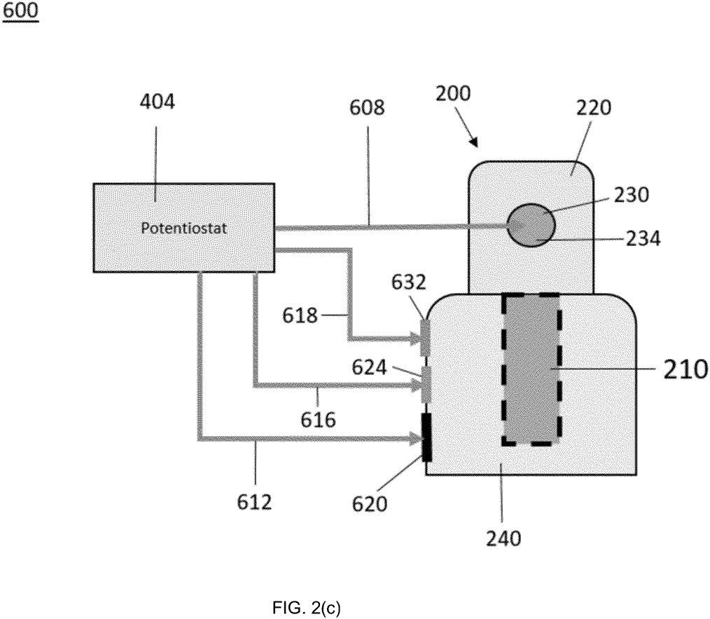

Referring to FIGS. 1(a) - 1(c), an exemplary dental implant 200 includes a metal abutment 208 and a dental crown 220, the latter being made from a non-conductive material such as ceramic, porcelain or a suitable polymer having an open end and an enclosed hollow cavity 224 that is suitably sized and shaped to be fitted over the metal abutment 208. The abutment 208 is tied to a metallic post 210, the latter component being shown more clearly in FIGS. 2(a) - 2(c), wherein the metallic post 210 is implanted directly into the jawbone 240 of the patient. According to this embodiment, the metal abutment 208 is press fit into the crown 220, the latter having an integrated metallic core 230 made in accordance with a specialized embodiment. The metallic core 230 is made from a suitable metallic material that enables and facilitates electrical conduction. In a preferred embodiment, the metallic core 230 is composed of a biocompatible metal commonly used in dentistry, such as but not limited to titanium, stainless steel and/or their alloys. When the metal abutment 208 is surgically implanted, the abutment 208 is placed into direct physical contact with a distal end of the metallic core 230, thereby creating an electrical connection. The metallic core 230 can alternatively be made integral with the abutment 208 (or can be formed as part of the crown 230), according to this embodiment.

More specifically and according to this specific embodiment, the metallic core 230 extends upwardly, as shown more specifically in FIG. 1(b), through the hollow cavity 224 of the dental crown 220, including a transverse portion of the core 230 that further extends to an opening formed in a side wall of the dental crown 220. A proximal end of the metallic core 230 is preferably flush with the side wall of the dental crown 220, thereby exposing a small metallic surface zone 234 on the dental crown 220, as shown. The shape of the exposed metallic surface zone 234 is circular according to this specific embodiment, but it will be understood that the shape of the defined zone 234 and the metallic core 230 can be suitably varied. Making the proximal end of the core 230 flush to the surface of the crown 220 is preferable so not to produce an overhang or create sharp edges. According to one version, the exposed surface area 234 of the metallic core 230 may be dimpled to allow for better mating with the crown attachment mechanism. In a preferred embodiment, the exposed metallic surface zone 234 resides on the inner facing wall of the crown 220 relative to the patient, such that the exposed metallic surface zone 234 is not visible.

The overall shape and configuration of the metallic core 230 can be suitably varied provided that the dental crown 220 can adequately and structurally function primarily as a prosthetic tooth. For example and in lieu of extending transversely as shown, the proximal end of the metallic core 230 can extend vertically through the hollow cavity 224 until exposed at a top surface of the crown 220. Other similarly based versions of implants that enable access to the metallic abutment and post, but without requiring removal of the crown are described in U.S. Pat. Application No. 16/884,664, which is incorporated herein by reference. As noted, this implant design provides a great advantage, when compared to other technologies, because the crown does not have to be removed in order to perform treatment.

Various systems are shown schematically in FIGS. 2(a) - 2(c) that can be utilized for treatment of an infected dental implant, such as implant 200, based on the application of a suitable cathodic stimulation voltage to the exposed metal surface area 234 without having to first remove the crown 220 to disrupt biofilm layers on the implant. The overall principles of this form (CVCES) of treatment are described in U.S. Pat. No. 9,616,142, previously incorporated herein in its entirety. As shown in FIGS. 2(a) - 2(c), the herein described dental implant 200 or other dental implants can be treated in conjunction with various CVCES treatment configurations or systems, herein labeled 400, 500, and 600, respectively. For purposes of this discussion, the dental implant 200 is shown schematically in use with each treatment system 400, 500, and 600. It will be understood that other metallic dental implants can be similarly treated using any of these exemplary treatment systems.

Each CVCES treatment system 400, 500 and 600, as shown diagrammatically in FIGS. 2(a) - 2(c), respectively, commonly includes a potentiostat 404 or similar device that is capable of generating an electrical potential as well as a number of electrodes, minimally including a working electrode and a counter electrode. The minimal configuration is shown with reference to FIG. 2(a), for a first exemplary CVCES treatment system 400, employing a pair of electrodes; namely, a working electrode and a counter electrode 420. The working electrode is the dental implant 200 based on the availability of the exposed metallic surface area 234 of the crown 230, while the counter electrode 420 is preferably made up of carbon, although other materials can be used. The counter electrode 420 is attached to the gum/jawbone 240 area of the patient via an electrical lead or wire 412 coupled to the potentiostat 404. An electrical lead 408 is further provided extending to a conductive member that is placed into contact with the exposed metallic area 234 of the crown 230. Electrochemical current is caused to flow based on voltages applied by the potentiostat 404 via an electrochemical circuit being formed between the working electrode 200 and the counter electrode 420. Due to the exposure area 234, the potentiostat 404 is electrically connected to the metal abutment 208, and thus the entire post-abutment-core system is electrically connected. However, the only metallic materials that are in contact with a conductive electrolyte (bone and soft tissue) are the metal abutment 208 and the post 210. Because the metallic core 230 is encapsulated by the non-conductive crown material, the metal core 230 essentially acts as an electrical wire or lead capable of transferring electrical energy (i.e., current) from the potentiostat lead to the dental implant 200. This system 400 provides a significant advantage, when compared to other comparable potentiometric treatment systems or techniques, because the dental crown 220 does not have to be first removed in order to perform treatment.

FIG. 2(b) diagrammatically illustrates a three (3) electrode system 500 that includes the working electrode (the implant 200), as well as a counter electrode 520 functioning in the same manner as that of the prior system 400. Each electrode 200, 520 is coupled to the potentiostat 404 via electrical leads 508 and 512, respectively. A third (reference) electrode 524 is applied along with the counter electrode 520 to the gums/jawbone area 240 of the subject and is electrically coupled to the potentiostat 404 via a corresponding lead 516. The reference electrode 524 permits greater electrochemical control for the treatment system 500. In a preferred embodiment, the reference electrode 524 is made from silver/silver chloride, thus producing a stable electrochemical biopotential for the working electrode (implant 200). Further details relating to the functioning of the potentiostat and counter and reference electrodes of this system 500 are described in greater detail in U.S. Pat. No. 9,616,142, previously incorporated by reference in its entirety.

FIG. 2(c) diagrammatically illustrates yet another version of a CVCES treatment configuration 600 that employs four (4) electrodes. As in the preceding, the dental implant 200 due to the exposed metallic area 234 acts a working electrode as electrically coupled to the potentiostat 404 by electrical lead 608. A counter electrode 620 and reference electrode 624 are attached to the gum/jawbone area 240 of the patient as connected to the potentiostat 404 by leads 612 and 616, respectively. Each of the electrodes 620 and 624 operate in the same manner as those previously described. In addition, this treatment system 600 is further equipped with a working sense electrode 632, similarly attached to the gum/jawbone 240 area of the patient and coupled to the potentiostat 404 by electrical lead 618. The working sense electrode 632 enables further control and data feedback of the working electrode (implant 200). Other suitable cathodic voltage treatment configurations or systems can also be utilized. In addition and though the counter, reference and working sense electrodes are shown according to this embodiment being attached to the jawbone/gum area 240, FIGS. 2(a) - 2(c), of the patient, other suitable positioning of these electrodes is permitted. For example, any or all of these electrodes could also be located outside the mouth on the face or completely external to the body and connected via a salt bridge.

Advantageously, each of the above systems/configurations permit reliable treatment of the dental implant 200, but without requiring removal of the crown. The exposed metallic surface area 234 of the crown permits electrical conduction to the remainder of the dental implant. Exposed metal surfaces are both safe and cosmetically acceptable when applying these designs and embodiments. A distinct differentiator from alternative dental treatment techniques is that the herein described implant promotes conduction over the entire bone-embedded surface of the dental implant, and not just conduction, for example, within the abscess pocket. This differentiator is a significant advance, especially in regard to dental implant posts. Implant posts are typically manufactured with a very rough, coarse microsurface to promote osseointegration. One issue this microsurface can create is that bacteria are able to “hide” within the crevices of the microstructure, even when bone matrix are apparently grown into the surface. The approach and design of herein described apparatus allows for thorough treatment of all microstructures in the metal, even with bone present, to eliminate all bacteria from those locations. It has been found and substantiated in scientific literature that at optimized treatment parameters, matrix embedded bone cells that are local to the reaction are not affected to a high degree.

Apparatus are now described in accordance with a number of embodiments according to various aspects of the present invention for use in a treatment system. More specifically, the following described apparatus can be used in connection with treatment systems for enabling electrical contact with the working electrode (metallic dental implant) and/or retaining the counter and/or reference electrode.

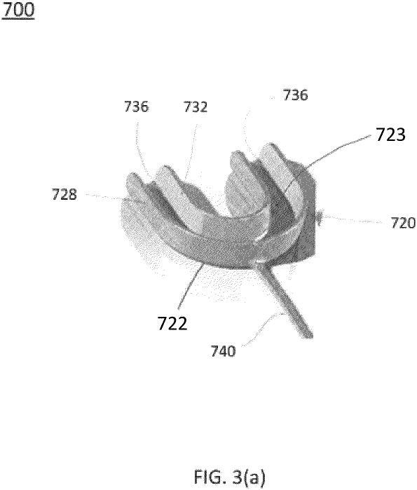

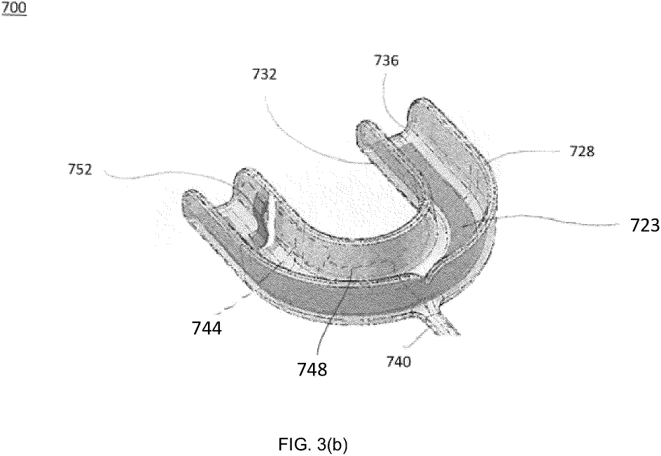

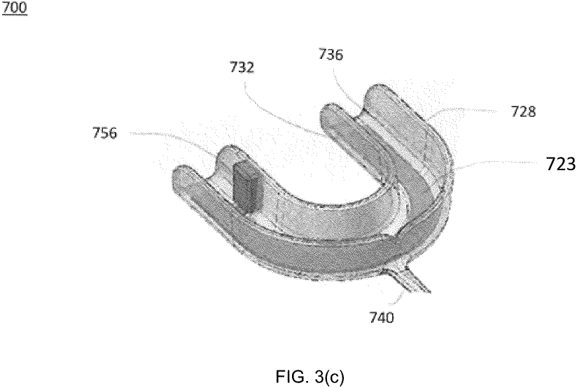

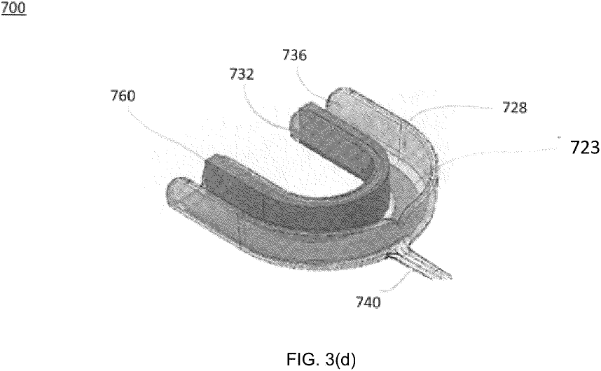

With reference to FIGS. 3(a) - 3(d), there is shown a first embodiment of an apparatus 700 that can used in conjunction with the CVCES treatment systems 400, 500, 600, FIGS. 2(a) - 2(c), as well as other systems designed to remove biofilms from metallic surfaces. The herein described apparatus 700 is described by way of example for use with the dental implant 200, FIGS. 1(a) - 1(c), having the exposed metallic surface area 234, FIGS. 1(a) - 1(c), on the crown 220 to enable direct electrical connection to the metal abutment/post of the dental implant 200. It will be understood, however, that the specialized dental implant 200 is merely discussed as an example wherein the described apparatus 700 is suitable for use with other dental implant designs.

As previously described and in a CVCES treatment system such as system 500, FIG. 2(b), the dental implant 200 is employed as the working electrode and the cathode of the formed electrochemical cell. The herein described apparatus 700 is a connective body that is configured to provide electrical engagement between an external electrical source such as a potentiostat 404, FIG. 2(b), and the dental implant 200. More specifically and according to this embodiment, the herein described apparatus 700 is defined by a semi-circularly shaped mouth guard 720. The mouth guard 720 is made from a moldable biofriendly thermoplastic or other suitable structural material that is sized and configured to fit within the mouth of a patient. More specifically and according to this embodiment, the mouth guard 720 includes an outer side or surface 722 and an opposing inner side or surface 723. A front or outer circumferential section 728 and a rear or inner circumferential section 732 each project from the inner surface 723, each of the front and rear circumferential sections 728, 732 being separated by an open-ended circumferential recess 736. The recess 736 is defined by a height and width dimension enabling the mouth guard 720 to be placed over the teeth (not shown) of a patient in either the entire upper or lower portion of the mouth. Alternative mouth guard designs are contemplated for purposes of this invention. For example, a single mouth guard can be sized and configured to cover both the upper and lower sets of teeth of a patient at the same time.

According to this embodiment and as shown in FIG. 3(b), the mouth guard 720 is custom formfitted to the patient and includes at least one metal contact 752 integrated in a wall of at least one of the front and rear circumferential sections 728, 732. The location of the at least one metal contact can be determined during the manufacturing process of the mouth guard 720 and is patient specific, depending on the location of the implant(s). Since the apparatus 700 is patient specific, a single metal contact can be provided or the apparatus 700 can include two (2) or more metal contacts for use with patients having multiple infected dental implants.

In terms of electrical connection and still referring to FIG. 3(b), the at least one metal contact 752 is coupled to a grounding plate 744 imbedded within the wall of the mouth guard 720, the latter grounding plate 744 being coupled by to an imbedded wire 748 that extends to an extending electrical lead 740 at one end of the mouth guard 720. The extending electrical lead 740 is preferably coated with a polymer or other insulating material and configured for attachment to an external electrical supply, such as a potentiostat 404, FIGS. 2(a) - 2(c).

According to at least one version, the mouth guard 720 may be reusable or alternatively could be designed as a single patient or single use apparatus. According to a preferred embodiment, the at least one metal contact 752 is biasedly positioned on the interior surface (i.e., the surface facing the circumferential recess 736) of either the outer or inner circumferential sections 728, 732 of the mouth guard 720 to promote electrical contact with the exposed metal area 234, FIG. 1(b), of the dental implant 200.

Various means for biasing the at least one metal contact 752 of the mouth guard 720 can be employed. For example, the at least one metal contact 752 can be spring loaded relative to one of the circumferential sections 728, 732 of the mouth guard 720. Alternatively, biasing can be provided by manufacturing the at least one metal contact 752 from a section of a spring steel, as shown in FIG. 3(b).

Alternatively and as shown in FIGS. 3(c) and 3(d), the at least one metal contact can be made from a conductive sponge 756 or a section of steel wool 760, each of the latter materials inherently providing a spring-like quality, albeit a weaker one than those provided by either spring loading or manufacturing the at least one metal contact from a section of spring steel. Each of the foregoing contacts 752, 756, 760 are electrically coupled to the grounding plate 744, FIG. 3(b), and imbedded wire 748, FIG. 3(b), disposed within the mouth guard 720 wherein each or any of the foregoing techniques can be used for insuring that the at least one spring contact 752, 756, 760 is biased into contact with the exposed metal area 234, FIG. 1(b), of the dental implant 200, FIGS. 1(a) - 1(c). As noted, the grounding plate 744 and imbedded wire 748 provides a suitable electrical connection to the coated electrical lead 740, partially shown, leading to an external voltage supply of a treatment system, for example CVCES treatment systems 400, 500, 600, FIGS. 2(a) - 2(c), each having a potentiostat 404 or other suitable device that is capable of providing a cathodic stimulation voltage.

When using a conductive sponge or steel wool 756, 760 as a metal contact as shown in FIGS. 3(c) and (d), respectively, the contact components should be made as condensed as much as possible within the defined recess 736 without sacrificing contactability to the abutment or crown of the dental implant in order to reduce extra metallic surface area involved in the working electrode reaction. In addition, all of the contact components described according to this embodiment may be coated with an insulating polymer that exposes only the points of contact needed for electrical connection in order to reduce extraneous metal surface area.

The at least one electrical contact, as described according to this embodiment and having embedded contact points, allows for treatment of a dental implant without having to remove the crown. As previously discussed, removing the crown is an option that many dentists prefer not to perform because the dental crown may break or cause extra trauma to the afflicted tissue. In the case where a biofilm exists on the post and abutment, the flow of electrons into the bulk metal, out the metal surface, and into the electrolytic environment, will create bactericidal chemical species that attack the biofilm from the metal surface outwards. pH is also a large factor in the bactericidal effect as laboratory testing has shown that microenvironment pH levels microns away from the surface can reach an alkaline level of 12 within minutes of electrical stimulation.

Alternatively and for dental implants in which the abutment of the dental implant is not exposed or the crown does not provide an exposed electrical contact point, the contact mechanism embedded in the mouth guard 720 may alternatively include a needle (not shown), the latter being appropriately sized and configured to pierce the tissue and contact the abutment of a metal dental implant directly.

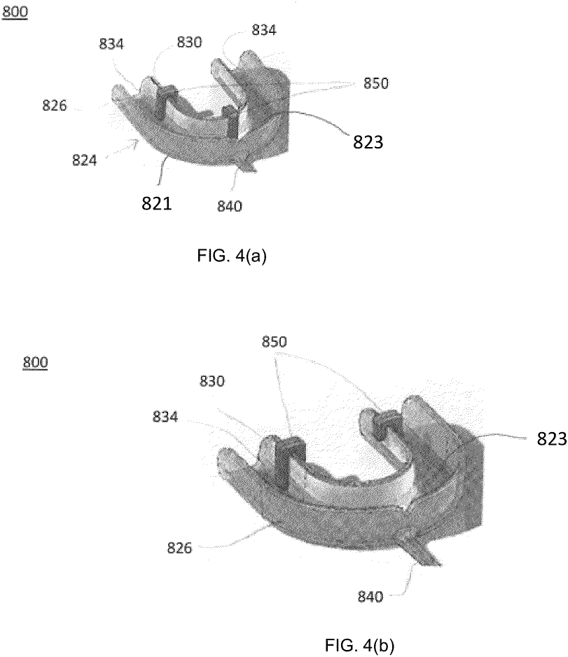

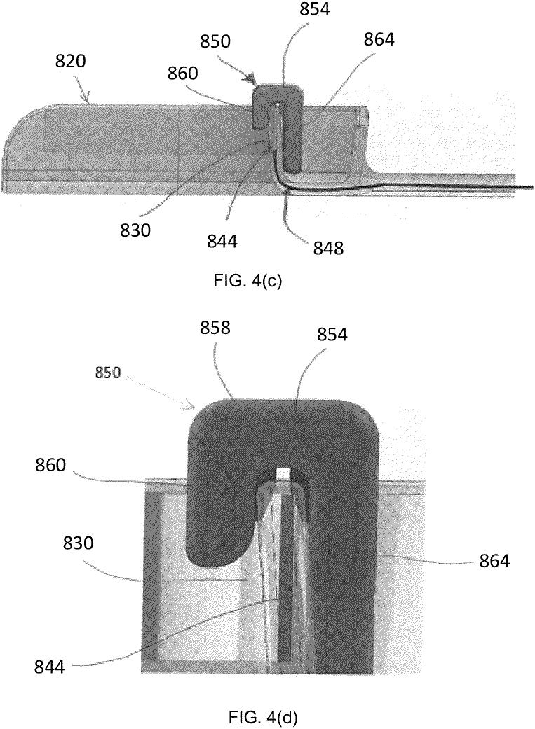

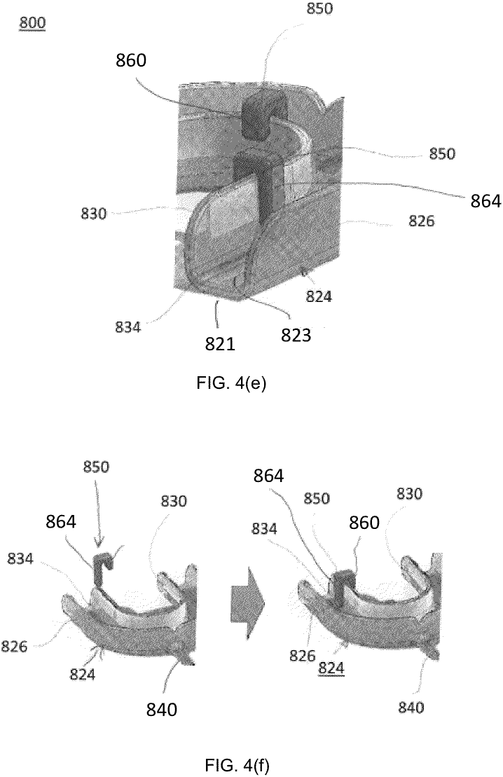

With reference to FIGS. 4(a) - 4(f), another exemplary apparatus 800 is described. The apparatus 800 according to this embodiment is defined by another connective body and more specifically by a generic mouth guard 824, which like the preceding described custom or formfitted version 700, FIGS. 3(a) - 3(d), has a semicircular shape or configuration. The mouth guard 820 includes an outer facing side or surface 821, an opposing inner side or surface 823, as well as an outer or front circumferential section 826, an inner or rear circumferential section 830, and an open-ended circumferential recess 834, each extending from the inner surface 823. The recess 834 has a width and height dimension that enables the mouth guard 820 to be placed over the teeth and gums of the patient. Preferably, the mouth guard 820 is made from a moldable thermoplastic material. According to one version, the apparatus 800 can be cleaned or recycled for reuse. In another version, the apparatus 800 is designed for single patient or single use.

As shown in FIGS. 4(a), 4(c) and 4(d), the herein described apparatus 800 further contains a metal grounding plate 844, preferably made from sheet metal, which is embedded within the mouth guard 820, as well as an embedded wire 848 further extending to an electrical lead 840, the latter extending from the mouth guard 820 to an external voltage supply 404, FIGS. 2(a) - (c), of a CVCES implant treatment system 400, 500, 600, FIGS. 2(a) - (c). Preferably, the extending electrical lead 840 is coated with a protective polymeric or other suitable insulating layer.

According to this specific embodiment and rather than specifically integrating at least one electrical contact, at least one releasably attachable contact 850 is configured for placement over one of the inner and outer circumferential sections 826, 830 of the mouth guard 820. As shown in FIGS. 4(c) and 4(d), the at least one releasably attachable contact 850 is fabricated in the form of a clip-like member defined by a base portion 854, as well as pair of arm portions 860, 864 extending in parallel relation from the base portion 854 with a spacing 858 being defined between the arm portions 860, 864 that is sized to seat the releasable attached contact 850 over the outer or inner circumferential portion 826, 830 of the mouth guard 820. More specifically, the arm portion 864 is longer than the other arm portion 860 and further configured, when attached, to extend into the recess 834 of the mouth guard 820. The exterior side of the arm portion 864 is further configured with a contact portion, the latter being configured for engaging the dental implant.

As further shown in FIG. 4(d), the releasably attachable contact 850 according to this embodiment has embedded metal on the interior surfaces of each of the arm portions 860, 864 to “bite” into the sheet metal of the grounding plate 844 imbedded in the mouth guard 820.

Advantageously and according to this embodiment, the number and location of releasably attachable contacts 850 can be varied as needed in order to create alignment with an implant, irrespective of the implant’s location in the mouth of the patient. Again referring to FIGS. 3(a) - (c), the contact mechanism according to this embodiment may be spring loaded, spring steel/leaf-spring, or comprise a section of a conductive sponge or steel wool to create biasing, each enabling electrical contact with the exposed metal area 234, FIGS. 1(a) - 1(c), of the dental implant 200. Alternatively and in lieu of a contact, the leg portion 864 can be separately provided with a needle (not shown) extending therefrom and configured and positioned to directly engage the tissue below the gum line for contact with the abutment/post of the implant.

The ability to attach the herein releasable attachable contact(s) 850 to one of the wall sections 826, 830 of the mouth guard 820 provides considerable versatility, enabling placement of the contact(s) in literally any portion of the mouth of a patient as shown in FIGS. 4(a), (b) and (e), without the need for pre-molding a custom guard. However, in the case in which the dentist wishes to have no mouth guard in order to use conjunctive irrigational therapies to disrupt the biofilm, alternative embodiments of the working electrode contact are described that attach directly to the crown and without the need for a fitted mouth guard.

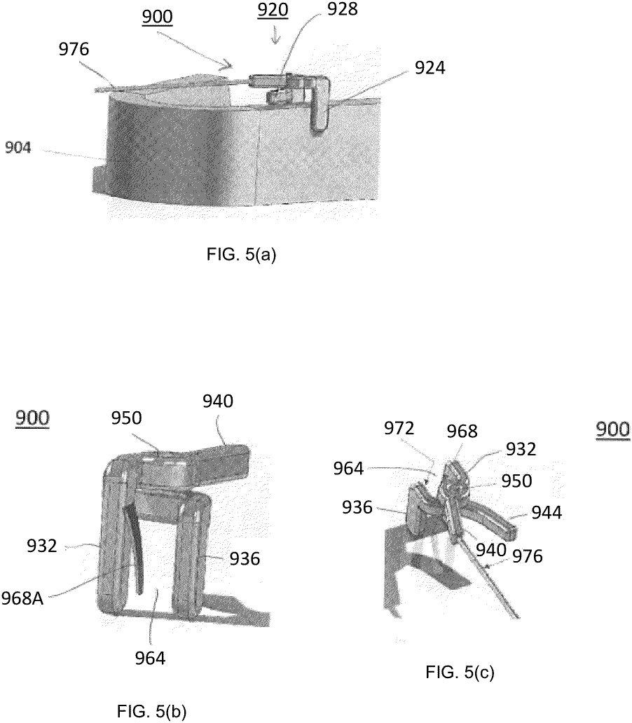

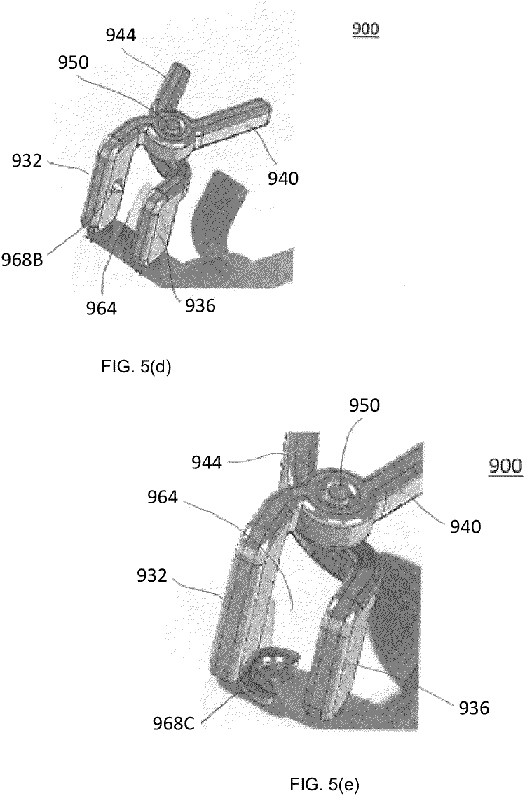

FIGS. 5(a) - 5(e) illustrates an apparatus 900 made in accordance with aspects of the invention to a connective body that can be used without an intermediate mouth guard. More specifically, the apparatus 900 is a spring loaded clip 920 having first and second half portions 924, 928. Each of the half portions 924, 928 commonly include a leg portion 932, 936, respectively, vertically extending from a distal end of each half portion 924, 928. An arm portion 940, 944 outwardly and transversely extends from an upper end of each leg portion 932, 936 in a curved configuration wherein each of the arm portions 940, 944 inwardly extend and then cross with one of the arm portions 944 being configured beneath the other arm portion 940. A torsional spring 950 provided on the upper arm portion 940 is coupled to the remaining lower arm portion 944 at the junction between the curved arm portions 940, 944. The torsional spring 950 according to this embodiment biases the leg portions 932, 936 of the apparatus 900 and more specifically defines a spacing 964 between the leg portions 932, 936 that can be further opened by inwardly squeezing the proximal ends of the arm portions 940, 944 to enable the apparatus 900 to be releasably secured over the teeth and gums of a patient, the latter being schematically shown as 904 in FIG. 5(a).

Referring to FIGS. 5(a) and 5(c), the legs 932, 936 include inwardly faced surfaces at each side of the defined spacing 964. The inwardly facing surface of leg portion 932 is provided with a metal contact 968 with the inwardly facing surface of the remaining leg portion 936 being provided with a soft pad 972, that is adhesively or otherwise attached. The metal contact 968 is directly wired through an imbedded wire (not shown) extending through the half section 924 to a proximal end of the arm portion 940, and further extending as an electrical lead 976, shown only in FIGS. 5(a) and 5(c), which is configured to make an electrical connection to an external voltage supply, such as potentiostat 404, FIG. 2(b), of the treatment system 500, FIG. 2(b).

According to this exemplary embodiment, the metal contact 968 is configured to make electrical contact with the exposed metal area 234 of the dental implant 200, FIGS. 1(a) -1(c), while the soft pad 972 on the remaining leg portion 936 of the apparatus 900 is preferably made from silicon, to help grip the tooth. As shown in FIGS. 5(b) and 5(d), the metal contact could be formed as a cantilevered section of spring steel 968A, as shown in FIG. 5(b), or a spring loaded pin 968B, as shown in FIG. 5(d). Alternatively, the metal contact 968 could also be made from a conductive sponge material or steel wool such as those described in prior embodiments, each of which is preferably biased to make an electrical connection with a dental implant, such as 200, FIGS. 1(a) - 1(c), or otherwise the clip member 900 could be configured with a needle (not shown) positioned to engage the dental implant below the gum line. According to another alternative version shown in FIG. 5(e), a half circular clip 968C on the lower end of the leg portion 932 may also be appropriate to clip on and contact the base of an abutment of an implant (not shown) that is very exposed. As in the preceding versions described, the apparatus 900 is preferably configured to provide connection to the working electrode of a CVCES treatment system, such as those shown in FIGS. 2(a) - 2(c), with the dental implant serving as the working electrode.

As previously discussed and in order to complete a circuit, there must at least exist one other electrode in a CVCES or other electrochemically based treatment system other than the metal dental implant, the latter acting as a working electrode, as previously shown in FIGS. 2(a), 2(b) and 2(c). In the case of a dental implant, a counter electrode of a CVCES treatment system ideally interfaces with the gums of the patient and more specifically, the gum in which the implant is implanted as opposed to a gum on the opposite side of the jaw or the other jaw entirely (upper or lower). Electrochemical current will flow between the implant and the counter electrode due to the conductiveness of the tissue. Though a two (2) electrode treatment system 400, such as shown in FIG. 2(a) will function, a two-electrode treatment system only allows for minimal control of the electrochemical processes on the working electrode because the potentials of the metal(s) of the implant can drift into thermodynamic regions that can cause corrosion or metal immunity. Accordingly, a treatment system having additional electrodes as shown in FIGS. 2(b) and 2(c) is preferable.

Though any number of electrodes can be used, a three-electrode system that includes an additional stable reference electrode is more favorable due to its balance of sufficient electrochemical control and number of electrodes that need to be in the mouth of the patient. In a preferred embodiment, the reference electrode is made from Ag/AgCl, thus providing a stable electrochemical biopotential for the working electrodes voltage to be referenced to. This function keeps the working electrode in safe electrochemical regions of thermodynamics. Although the counter electrode needs to interface with the gums to promote electrochemical current through the tissue to the dental implant, the electrode’s metal surface can exist either internal or external to the mouth as described herein.

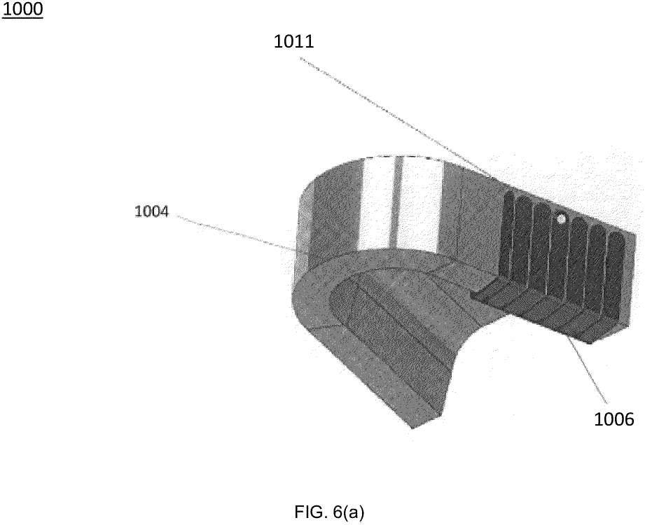

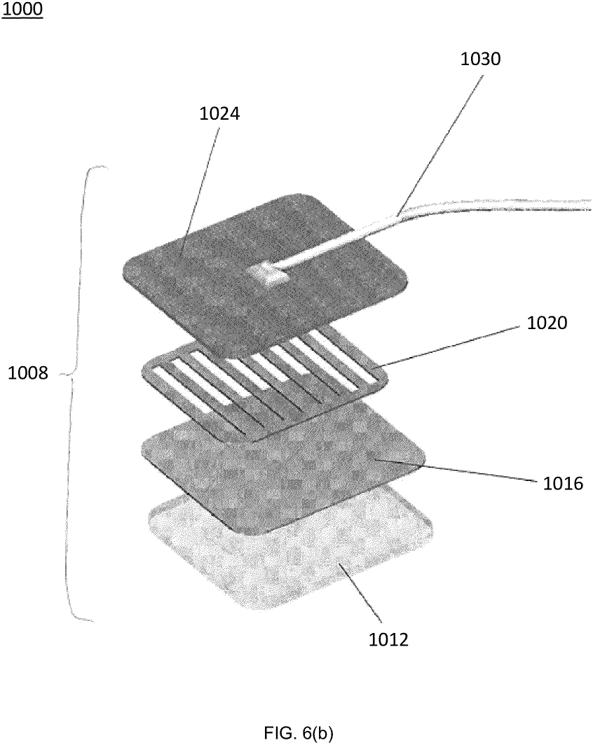

According to one version, shown in FIGS. 6(a) and 6(b), an exemplary apparatus 1000 is defined by a highly flexible connective body 1006 retaining a flexible electrode 1008 fabricated from a plurality of stacked layers that create an electrical connection. The connective body 1006 according to this embodiment is shaped and configured to be placed within the mouth of a patient and more specifically placed or wrapped over the teeth, with opposing sides of the flexible electrode 1008 being in contact with the gums of the patient.

According to this embodiment and as shown in FIG. 6(b), the flexible electrode 1008 is shown in an exploded form and includes a hydrogel layer 1012, which is preferably carbon backed with at least one buffering agent, an anodic conductive film layer 1016, a conductive mesh layer 1020, and an exterior layer 1024. The exterior layer 1024 is preferably being made from a flexible material, such as a fabric, that is sized and configured to surround the assembly 1000 and further includes an exterior adhesive that permits flexible attachment over the teeth and gums 1004 of the patient. An electrical lead 1030 extends from a stimulation device such as a potentiostat 404, FIG. 2(b), and attaches through the back of the exterior layer 1024. The conductive mesh layer 1020 behind the conductive anodic (preferably carbon) layer 1016 spreads the point of contact over a considerably larger area, wherein the mesh layer 1020 is preferably made from copper or platinum. The carbon film layer 1016 behind the buffered hydrogel layer 1012 acts as the conductive electrode surface for the reaction. Layer 1016 can alternatively be made from platinum, or other suitable metal that is chemically stable under anodic reactions. Additional details relating to this assembly are described in copending U.S. Pat. Application Serial No. 62/984,332, the entire contents of which are incorporated by reference.

In a preferred embodiment, the flexible connective body 1006 of the apparatus 1000 interfaces with the gums on both the inner and outer sides of the jaw around the implant, though in a less preferred embodiment there may exist only one electrode on only one side of the implant. Preferably, the electrode 1008 of the herein described apparatus 1000 is sufficiently flexible and may wrap over the teeth to adhere to both sides of the gum, or alternatively exist as two (2) separate electrodes that adhere to both sides of the gum, but are electrically connected to one another. Having the flexible electrode 1008 on both sides of the gums creates a more evenly distributed treatment on the dental implant itself. The electrode 1008 may incorporate flexible segments that contour more effectively to the jaw. The anodic reaction will build up an acidic pH within the hydrogel, and thus the starting pH of the hydrogel will be neutral or basic, preferably with a pH between 6 and 11. Preferably, the surface area of the electrode 1008 should be at least the same as the surface area of the dental implant to promote a more optimized treatment. This embodiment is directed to the counter electrode of a CVCES treatment system, such as treatment system 500, FIG. 2(b), but may also contain the reference electrode, herein shown as 1011 that can be incorporated into the flexible connective body 1006. The reference electrode 1011 may alternatively be provided as a separate electrode adhered to the gums, as opposed to being built into the herein described counter electrode while maintaining electrical isolation between the carbon and the Ag/Ag/Cl metal surfaces of the counter and reference electrodes, respectively.

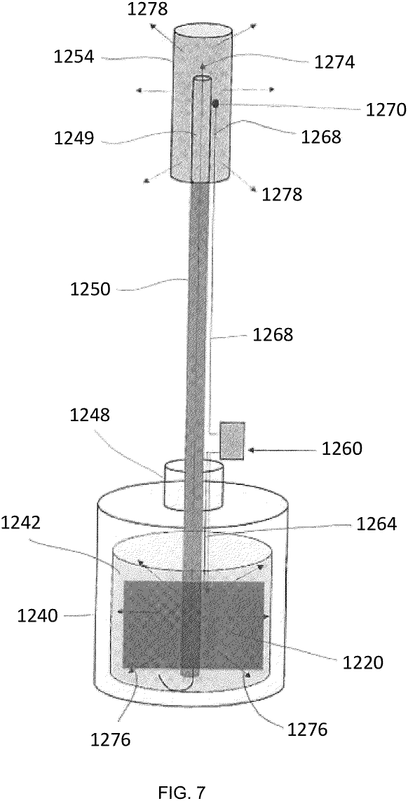

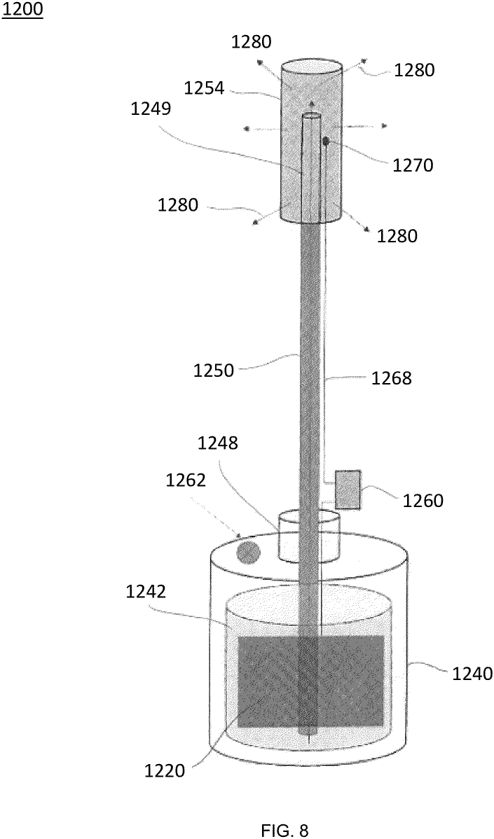

The foregoing described an apparatus used in connection with a CVCES or other suitable treatment system, such as treatment systems 400, 500, 600, FIGS. 2(a) - 2(c), with the counter electrode being disposed within the mouth of the patient proximate the dental implant. As noted above, the counter electrode of the treatment system can alternatively be disposed external to the mouth of a patient. As shown in FIGS. 7 and 8, an external electrolytic system 1200 is represented, the system 1200 including a metal counter electrode surface 1220 supported within a container 1240 or cartridge that is external to the mouth of the patient. The container 1240 is shaped and configured to retain a suitable quantity of a conductive fluid 1242, such as a salt solution, for enabling electrochemical current transport to the gum interface. In a preferred embodiment, the salt solution would be composed of sodium chloride and water; however, the salt solution may contain any suitable electrolytic salt compound that can be safely maintained within the oral cavity. According to one version, the container 1240 retains approximately 100 mL of conductive fluid. In other embodiments, for example, the container 1240 can be configured and sized to retain between 20 mL and 10 liters of conductive fluid, though it will be apparent that the amount of a retained conductive fluid 1242 can be easily varied.

According to this embodiment and in order to create an electrolytic bridge, the conductive fluid 1242 exits the container 1240 through a lid 1248 via a hollow tube 1250 having one proximal end extending through the lid 1248 and into the interior of the container 1240. The electrode 1220 according to this embodiment is defined by a carbon sheet or a platinum mesh that is fully or substantially immersed within the conductive fluid 1242 retained in the container 1240. The conductive fluid 1242 according to this embodiment can possess any pH and can be mildly acidic (pH of about 5.0), but preferably is neutral to basic and even more preferably is basic in pH in order to counteract the acid generation during treatment.

According to this embodiment, at least one cotton roll 1254 is disposed at a distal end 1249 of the hollow tube 1250. The at least one cotton roll 1254 can preferably be made from traditionally used dental cotton that often lines the gums for dental procedures. Alternatively, the roll 1254 can be made from synthetic cotton or other similar material. In a preferred embodiment, the distal end 1249 of the tube 1250 inserts into an end of the cotton roll 1254, as opposed to the cotton roll 1254 being inserted into the opening of the hollow tube 1250. A single cotton roll 1254 is shown for illustrative purposes, but it will be understood that one or more cotton rolls can be used. According to a preferred embodiment, the tube 1250 can be bifurcated with tube portions being separately attached to two cotton rolls that are disposed within the oral cavity of a patient.

The conductive fluid 1242 can be caused to flow from the container 1240 to the cotton roll(s) 1254 via the hollow tube 1250 by various means. For example, the container 1240 can be made from a flexible material that can be squeezed. According to another version, the container 1240 can be pressurized during its manufacture and provided with a seal (not shown) that can be broken by the dentist or physician/caregiver prior to use. In another version, the container 1240 can be provided with a one-way valve 1262, FIG. 8, permitting the user to push air into the container 1240 in order to displace conductive fluid 1242 through the attached tube 1250 to the cotton roll(s) 1254. According to yet another version, the container 1240 can be suspended or hung above the head of the patient wherein gravity can be used in order to feed the conductive fluid 1242 to the cotton roll(s) 1254. The foregoing are merely examples, as it will be readily understood that other suitable means configured for moving conductive fluid 1242 from the container 1240 to the cotton roll(s) 1254 can be employed.

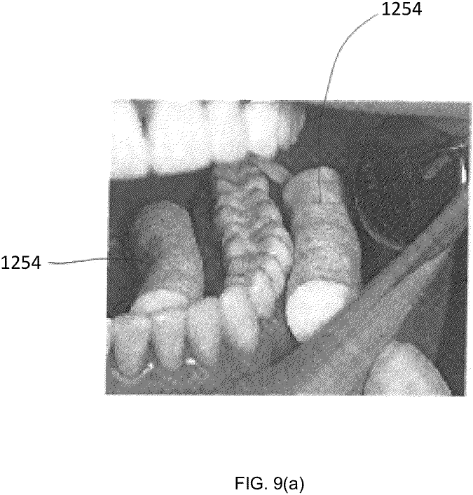

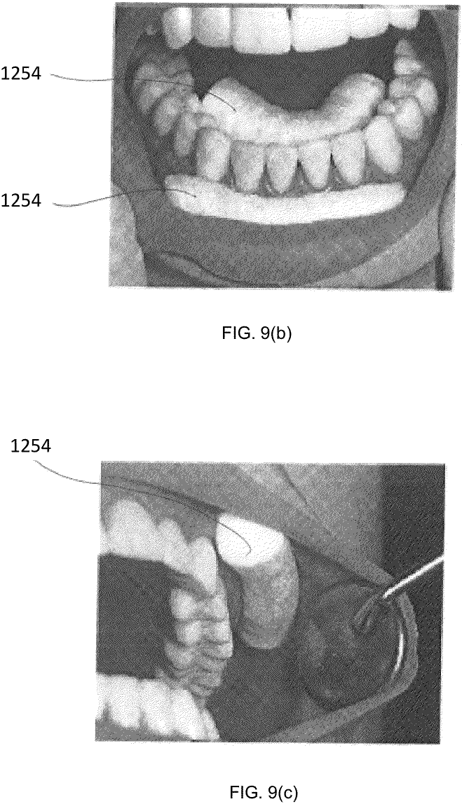

FIG. 7 schematically depicts the flow of current through the external electrolytic system 1200. Lines 1264 represent current flow that transports electrically from the external voltage supply, such as potentiostat 404, FIG. 5(b) of a treatment system, to the metal electrode surface 1222 (counter electrode) disposed in the fluid container 1240 via a connector, shown diagrammatically as 1260. The current then is converted to electrochemical current through faradaic and non-faradaic reactions and enters the conductive fluid 1242, shown as arrows 1276. The current can then shuttle via the electrolyte through the hollow tube 1250, into the at least one saturated cotton roll(s) 1254, and into the gum interface as shown by arrows 1278. The cotton roll(s) 1254 are retained at the gum interface of a patient, as shown in FIGS. 9(a) - 9(c) using an connective body disposed within the oral cavity of a patient in order to permit the conductive fluid 1242 to be directed to the gum interface. An example of a suitable connective body is described in U.S. Pat. No. 5,203,699, herein incorporated by reference in its entirety. The connective body according to the above patent is described for removing saliva from a patient in a dental procedure wherein the present apparatus is configured to support one and preferably two (or more) cotton rolls in a frame that is configured to positively engage and receive the conductive fluid (and current) to the gum interface for purposes of implant treatment.

As noted, it is preferable that a three-electrode treatment system or configuration such as shown in FIG. 2(b) be utilized. Regarding the herein described external electrolytic system embodiment, it is highly preferred that a stable reference electrode 1270 such as Ag/Ag-Cl stays internal to the mouth to be as close to the working electrode (e.g., the dental implant) as possible. High resistance between the working electrode and the reference electrode 1270 can cause marked drops in treatment current. Therefore, the reference electrode 1270 can be disposed as a typical adhesive hydrogel electrode to the gum line or alternatively be incorporated into the body of the cotton roll 1254 as shown schematically in FIGS. 7 and 8. In each of the foregoing arrangements, the reference electrode 1270 is separately coupled via lead 1268 to the external voltage supplying device (potentiostat 404, FIG. 2(b)) of the treatment system 500, FIG. 2(b) through the connector 1260.

The fluidic configuration of the external system 1200 is shown in FIG. 8. An electrode applicator is configured to retain cotton roll(s) 1254 on opposing sides of a frame (not shown) and receive conductive fluid 1242 from the container 1240, saturating the cotton roll(s) 1254 and creating an electrolytic bridge with the gums of the patient and forming an electrochemical cell with the working electrode (metal dental implant). Once an amount of conductive fluid 1242 is moved from the container 1240 and saturates the at least one cotton roll 1254, the cotton roll(s) 1254 is mechanically stabilized to the gum interface of the patient in a manner similar to that described with regard to the internally disposed electrode 1008, previously shown in FIGS. 6(a) and 6(b), including both the inner and outer sides of the gum. The continuous electrolyte bridge created and extending from the dental implant, through the gums, the at least one cotton roll(s) 1254, the conductive fluid 1242, and the metal surface 1222 enables this external electrolytic system 1200. As such, the herein disclosed embodiment is configured to supply the cotton roll(s) 1254 with a fluid in order to promote a conductive pathway to the metallic dental implant.

A main advantage of the foregoing external electrolytic system 1200, as compared to the version of FIGS. 6(a) and 6(b), is that acid build-up from the anodic reaction on the counter electrode can be mitigated to a much higher degree with an external metal surface 1220. The externally disposed metal surface 1220 can be provided with a much higher surface area than an internally disposed counter electrode because there is no size restriction, such as found in the mouth of the patient. The external metal surface 1220 may exist as a planar sheet, a conductive mesh, or alternatively as folded sheets in order to increase the surface area. With an increase in surface area, fewer faradaic chemical reactions are needed to take place to support the reaction at the working electrode and thus acid build-up is reduced. Also, the container 1240 of conductive fluid 1242 provides a much larger volume of electrolyte for the acid to diffuse into, and thus concentrations of acid per volume can be reduced. As noted, the conductive fluid 1242 also preferably exists as a neutral to basic pH to assist in neutralizing any acid build-up. However, one disadvantage of the described external system 1200 is that there now exists more electrochemical resistance between the electrodes due to distance and volume of fluid between the electrodes. This increase in resistance may cause losses in current and thus therapy strength to the biofilm layer of the implant(s) being treated. This challenge can be overcome by increasing the conductivity of the conductive fluid 1242, optimizing the overall distance of the hollow tube 1250 (thus reducing volume of fluid), maximizing the surface area size of the counter electrode sheet or mesh 1220, and ensuring that the electronics of the external power supply of the stimulation device of the treatment system, such as system 500, FIG. 2(b), contains suitable voltage limitations, such that the voltages required by the treatment reaction can be accommodated by the external power supply (potentiostat 404, FIG. 2(b)).

PARTS LIST FOR FIG. 1 - 9(c)

| | |
|---|---|
| 200 | dental implant |
| 208 | abutment |
| 210 | post |
| 220 | crown |
| 224 | hollow cavity, crown |
| 230 | metallic core |
| 234 | exposed surface area |
| 240 | jawbone/gums |
| 400 | treatment system |
| 404 | potentiostat |
| 408 | electrical lead |
| 412 | electrical lead |
| 420 | counter electrode |
| 500 | treatment system |
| 508 | electrical lead |
| 512 | electrical lead |
| 516 | electrical lead |
| 520 | working electrode |
| 524 | reference electrode |
| 600 | treatment system |
| 608 | electrical lead |
| 612 | electrical lead |
| 616 | electrical lead |
| 620 | working electrode |
| 624 | reference electrode |
| 632 | sense electrode |
| 700 | apparatus |
| 720 | custom mouth guard |
| 721 | outer side or surface |
| 723 | inner side or surface |
| 728 | front or outer circumferential section |
| 732 | rear or inner circumferential section |
| 736 | circumferential recess |
| 740 | electrical lead |
| 744 | grounding plate |
| 748 | imbedded wire |
| 752 | contact |
| 756 | conductive sponge |
| 760 | steel wool |
| 800 | apparatus |
| 820 | working mouth guard |
| 821 | outer facing side or surface |
| 823 | inner surface |
| 826 | front or outer circumferential section |
| 830 | rear or inner circumferential section |
| 834 | recess |
| 840 | electrical lead |
| 844 | grounding plate |
| 848 | imbedded wire |
| 850 | releasably attachable contact |
| 854 | base portion, contact |
| 858 | spacing, contact |
| 860 | leg portion, contact |
| 864 | leg portion, contact |
| 900 | apparatus |
| 904 | jaw line/ gums (patient) |
| 920 | torsional clip |
| 924 | half section, clip |
| 928 | half section, clip |
| 932 | leg portion |
| 936 | leg portion |
| 940 | arm portion |
| 944 | arm portion |
| 950 | torsional spring |
| 964 | spacing |
| 968 | contact, metal |
| 968A | cantilevered piece of metal |
| 968B | spring-loaded pin |
| 968C | half-circular clip |
| 972 | pad |
| 976 | electrical lead |
| 1000 | apparatus |
| 1006 | connective body |
| 1008 | electrode |
| 1012 | buffered hydrogel layer |
| 1016 | conductive layer |
| 1020 | conductive mesh layer |
| 1024 | exterior layer |
| 1030 | electrical lead |
| 1200 | external electrolytic system |
| 1220 | metal electrode surface |
| 1240 | container |
| 1242 | conductive fluid |
| 1248 | lid, container |
| 1249 | distal end, tube |
| 1250 | tube |
| 1254 | cotton roll(s) |
| 1260 | connector |
| 1262 | one way valve |
| 1264 | line |
| 1268 | line |
| 1270 | reference electrode |
| 1276 | arrows |
| 1278 | arrows |
| 1280 | arrows |

The preceding embodiments are examples and it will be understood to the reader that a number modifications and variations can be made in accordance with the present invention including the following claims. For example and though the embodiments have been described for use with specific electrodes, the various apparatus could be used in conjunction with other electrodes. For example, the internal mouth disposed counter electrode could also be optimal to use in conjunction with the torsional clip working electrode contacting mechanism.

1-22. (canceled)

23. A treatment system to disrupt bacteria from a metallic dental implant comprising:
a device capable of producing a cathodic stimulation voltage;
a working electrode that comprises the metallic dental implant;
a counter electrode, each of the counter electrode and working electrode being connected via a circuit to the device capable of producing a stimulation voltage, the counter electrode comprising:
a container retaining a conductive fluid and an electrically conductive member, the container being connected to the device capable of producing the stimulation voltage; and
a connective body fluidically coupled to the container and the gum interface of a patient in relation to the metallic dental implant, the connective body being configured to receive conductive fluid and current created by the device capable of producing the stimulation voltage.

24. The treatment system according to claim 23, in which the conductive fluid is neutral to basic in pH.

25. The treatment system according to claim 23, in which the container retaining the conductive fluid is connected to the connective body by at least one hollow tube.

26. The treatment system according to claim 25, wherein the connective body includes at least cotton roll configured to receive the conductive fluid from the container.

27. The treatment system according to claim 23, further comprising a reference electrode coupled to the circuit.

28. The treatment system according to claim 27, wherein the reference electrode is disposed on the connective body in relation to the metallic dental implant.

29-62. (canceled)

63. The treatment system according to claim 26, wherein the at least one cotton roll is disposed at an end of the at least one hollow tube opposite the container.

64. The treatment system according to claim 23, wherein the electrically conductive member is immersed into the conductive fluid retained in the container.

65. The treatment system according to claim 64, in which the electrically conductive member is a sheet made from one of carbon or platinum.

66. The treatment system according to claim 23, including means for moving conductive fluid from the container to the connective body.

67. The treatment system according to claim 66, wherein the means for moving conductive fluid comprises one of a gravity feed, a motorized pump, squeezing the container, and a one way valve.

68. The treatment system according to claim 28, further comprising a connector disposed between the container and the device capable of producing the stimulation voltage.

69. The treatment system according to claim 68, wherein the reference electrode includes a lead extending to the connector.

70. The treatment system according to claim 69, wherein the reference electrode is comprised of AgCl.
